(19) United States
(12) Patent Application Publication
(10) Pub. No.: US 2010/0055043 A1
(43) Pub. Date: Mar. 4, 2010

(54) Poly-Halo Metal X-ray Contrast Agents

(76) Inventor: Dennis A. Moore, St. Louis, MO (US)

Correspondence Address: Mallinckrodt Inc., 675 McDonnell Boulevard, HAZELWOOD, MO, 63042, US

(21) Appl. No.: 12/447,770

(22) PCT Filed: Oct. 30, 2007
(86) PCT No.: PCT/US07/22917

§ 371 Date: Apr. 29, 2009

Related U.S. Application Data
(60) Provisional application No. 60/855,304, filed on Oct. 30, 2006.

(51) Int. Cl. A61K 49/04; C07D 257/02

(52) U.S. Cl. 424/9.44; 540/474; 540/465

(57) ABSTRACT

In certain aspects, the present invention relates to metal coordinating complexes for use as imaging contrast agents. For instance, in some embodiments, the present invention is directed to an imaging contrast agent including a metal chelator and a halogen-substituted phenol, thiophenol, resorcinol, thioresorcinol, or dithioresorcinol derivative.

### BACKGROUND

The present invention is generally directed to imaging contrast agents. In particular, the present invention is directed to metal coordinating moieties that allow for the safe administration of a highly opaque class of metals, such as bismuth and lead.

The search for ideal contrast media for X-ray radiodiagnostic studies has extended over many decades. Bismuth subnitrate was the first radiocontrast agent used for visualization of the alimentary tract. Later, barium sulfate, a safer agent, was introduced. Barium sulfate has remained the most widely used radiographic agent for the alimentary tract (W. H. Strain, International Encyclopedia of Pharmacology and Therapeutics, Section 76, Vol. 1, Radiocontrast Agents, Chapter 1, Historical Development of Radiocontrast Agents, 1971, Pergamon Press). The inorganic, insoluble oral agents like bismuth subnitrate and barium sulfate serve as valuable tools for gastrointestinal radiodiagnosis.

Unlike gastrointestinal radiodiagnosis, urographic and angiographic X-ray procedures require intravascular administration of a safe, water-soluble, radiopaque contrast medium. Since the introduction of the water-soluble ionic triiodobenzoic acid derivatives, such as diatrizoic acid and iothalamic acid, in the early 1960's, radiographic visualization of the vascular system has become the most important application of X-ray contrast media. These X-ray procedures are valuable in the diagnosis and evaluation of a variety of diseases that involve or cause alterations in normal vascular anatomy or physiology.

The progress in X-ray contrast media development has been extensively documented; e.g., U. Speck, “X-ray Contrast Media”, Medical Division Publication, Department of Medical Information, Schering A G; D. P. Swanson et al., “Pharmaceuticals in Medical Imaging” (1990) McMillan Publishing Co.; M. Sovak, “Radiocontrast Agents”, (1984), Springer Verlag. Preferred intravascular X-ray contrast agents possess a combination of desirable properties. Such properties include the following to various degrees: (1) maximum X-ray opacity; (2) biological safety; (3) high water solubility; (4) chemical stability; (5) low osmolality; and (6) low viscosity. In particular, studies have shown that high osmolality can be correlated with undesirable physiologic adverse reactions to X-ray contrast media, e.g., nausea, vomiting, heat and pain.

A significant advancement in X-ray contrast media has been the development of nonionic triiodobenzoic acid derivatives such as iopamidol, iohexyl and ioversol. In general, aqueous solutions of these non-ionic agents have less osmolality than previous agents and hence, provide greater patient comfort when injected. Adverse reactions, especially in the sensation of pain, warmth, and hemodynamic effects are greatly reduced as compared to the ionic triiodobenzoic acid derivatives.

Further reduction of osmolality of X-ray contrast media resulted from the introduction of nonionic dimeric agents such as iotrolan and iodixanol. These agents, as compared to the nonionic monomeric agents, provide even greater patient comfort by reducing nausea and vomiting upon intravenous injection and by causing much less pain upon peripheral arterial injection. The viscosity of such nonionic dimeric agent-based formulations, however, is generally greater than for the corresponding monomeric analogs. Further, as a result of low osmolalities, ionic additives were required to achieve isoosmolality with blood. Thus there remains a need for optimized forms of triiodoaromatic monomers and dimers with low viscosity biological osmolality.

Despite the progress made over the years, there still exists a need for new X-ray contrast media that possess greater potency thereby allowing better visualization of the target tissue and organs, without sacrificing safety. The potency of X-ray contrast media can be described as its molar ability to absorb X-rays in vivo, thereby allowing the generation of clinically useful images. As stated above, current technology has focused on conventional approaches to iodinated aromatic species. These species, however, reach a practical opacity limit due to the safety and stability concerns resulting from the ratio of iodine to carbon. The imaging contrast agents of the present invention can be used with metals that are more X-ray opaque than iodine thereby improving visualization of target tissues and organs.

### SUMMARY

Among the several aspects of the present invention is the provision of imaging contrast agents for use in diagnostic procedures. Advantageously, imaging contrast agents of the invention may tend to exhibit greater potency than conventional non-ionic and dimer-like compounds, while maintaining the safety profile generally associated with these compounds.

One aspect of the present invention is directed to an imaging contrast agent that includes a metal chelator and a halogen-substituted resorcinol, thioresorcinol, or dithioresorcinol derivative. In some embodiments, the metal chelator may be complexed with a radioactive, paramagnetic or radiopaque metal.

Another aspect of the invention is directed to a method of medical imaging. In this method, an imaging contrast agent of the invention is administered to a patient. In some embodiments, the patient may be imaged before, during and/or after administration of the agent.

Other aspects of the invention will be in part apparent and in part pointed out hereinafter.

### DETAILED DESCRIPTION OF VARIOUS EMBODIMENTS

The present invention provides for imaging contrast agents that comprise a metal chelator and a halogen-substituted phenol, thiophenol, resorcinol, thioresorcinol, or dithioresorcinol derivative (sometimes the phenol and thiophenol groups are collectively referred to as (thio)phenol and sometimes the resorcinol, thioresorcinol, and dithioresorcinol groups are collectively referred to as ((di)thio)resorcinol). Together, the metal chelator and the halogen-substituted (thio)phenol or ((di)thio)resorcinol derivative are sometimes referred to as the “metal coordinating moiety”. The metal coordinating moiety can rapidly form coordination complexes with metals (sometimes referred to herein as “metal complexes” or simply “complexes”) for use in diagnostic metalloradiopharmaceuticals or as X-ray or magnetic resonance imaging contrast agents. Advantageously, the metal coordinating moieties are able to coordinate metals that are more opaque to X-ray than iodine, e.g., lutetium, lead, bismuth, and mercury. Thus, a lower dose may be given without loss of efficacy or potency. The lower concentration may also give rise to iso- or hypoosmolar formulations, depending on the final structure of the metal coordinating moiety.

As described above, the metal coordinating moiety of the present invention comprises a metal chelator and a halogen-substituted (thio)phenol or ((di)thio)resorcinol derivative. The halogen-substituted (thio)phenol or ((di)thio)resorcinol moieties of the present invention comprise a phenyl ring wherein the ring is substituted by at least two halogen atoms and by (a) one hydroxy group, (b) two hydroxy groups, (c) one thiol group, (d) two thiol groups, or (e) one hydroxy and one thiol group. The hydroxy and/or thiol group(s) are located at the ring carbon atom(s) alpha to the ring carbon atom at the point of attachment of the metal chelator. Further, the two carbon atoms beta to the carbon atom at the point of attachment of the metal chelator are substituted by halogen atoms. In addition, the carbon atom gamma to the carbon atom at the point of attachment to the metal chelator is optionally substituted with a group that influences stability, biodistribution and/or toxicity.

In one embodiment, the halogen-substituted (thio)phenol or ((di)thio)resorcinol) moiety of the present invention has the general Formula (1):

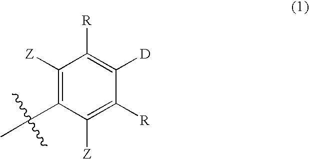

wherein

each Z is independently hydrogen, hydroxy, or thiol provided, however, that at least one Z substituent is other than hydrogen;

each R is independently bromo or iodo; and

D is hydrogen or a substituent selected to influence stability, biodistribution and/or toxicity.

Without being held to any particular theory, it is believed that the orientation of the hydroxyl and/or thiol group(s) of the halogen-substituted (thio)phenol or ((di)thio)resorcinol moiety at the ring carbon atom(s) alpha to the ring carbon atom at the point of attachment of the metal chelator offers a more robust coordination environment for the metal. In one embodiment, only one of the carbon atoms alpha to the carbon atom at the point of attachment of the metal chelator on the phenyl ring is substituted by a thiol or hydroxy group. In an alternative embodiment, both of the carbon atoms alpha to the carbon atom at the point of attachment of the metal chelator on the phenyl ring are independently substituted by a hydroxyl or thiol group. By way of example, it is known that yttrium-oxygen coordination bonds are quite labile. Thus, in solution this bond is breaking and reforming very rapidly. The availability of a second positionally equivalent, phenolic oxygen (in the case of a resorcinol derivative) allows for quick reformation of the oxygen-metal bond. Consequently, the second oxygen provides an intramolecular competitive binding event versus any external competition, which could lead to decomplexation and decomposition of the metal coordinating complex. Similarly, because many metals form stable coordination bonds with thiol groups, one or both of the hydroxyl groups may be replaced with a thiol group.

Prior to use in a diagnostic procedure, the metal coordinating moiety is complexed with a metal to form a metallopharmaceutical diagnostic agent of the present invention.

### Metals

Any metal capable of being detected in a diagnostic procedure in vivo or in vitro may be employed as a metal in the present conjugates. Particularly, any radioactive metal ion, paramagnetic metal ion, or x-ray opaque metal ion capable of producing a diagnostic result in a human or animal body or in an in vitro diagnostic assay may be used. The selection of an appropriate metal based on the intended purpose is known by those skilled in the art. In one embodiment, the metal is selected from the group consisting of W, Hg, Pb, Lu, Lu-177, Y, Y-90, In, In-111, Tc, Tc═O, Tc-99m, Tc-99 m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211. For example, the metal may be selected from the group consisting of W, Lu, Hg, Pb, Bi, Y-90, In-111, Tc-99m, Re-186, Re-188, Cu-64, Ga-67, Ga-68 and Lu-177. By way of further example, the metal may be selected from a more restrictive group, e.g., Y-90, In-111, Tc-99m, Re-186, Cu-64, Ga-67, and Lu-177 or Lu, Hg, Pb and W. In another embodiment, metals that form labile bonds with oxygen, such as yttrium and indium, are appropriate metals for metal coordinating moieties having a halogen-substituted (thio)phenol or ((di)thio)resorcinol moiety.

### Metal Coordinating Moiety

The metal coordinating moiety of the present invention may be any moiety having a halogen-substituted (thio)thiophenol or ((di)thio)resorcinol derivative used to complex, or coordinate, one or more metals under physiological conditions. Preferably, the metal coordinating moiety forms a thermodynamically and kinetically stable complex with the metal to keep the complex intact under physiological conditions; otherwise, systemic release of the coordinated metal may result.

As previously stated, the metal coordinating moiety comprises two components, (a) the metal chelator and (b) the halogen-substituted (thio)phenol or ((di)thio)resorcinol derivative. Although not required, the oxygen or sulfur atom(s) comprising the hydroxyl or thiol group(s), respectively, of the halogen-substituted (thio)phenol or ((di)thio)resorcinol derivative may participate in the complexation of the metal. In other words, the metal coordinating moiety may complex the metal with or without the participation of the hydroxyl or thiol group(s) of the halogen-substituted (thio)phenol or ((di)thio)resorcinol derivative. The participation of these hydroxyl or thiol group(s) will depend upon the nature of the metal chelator and the particular metal selected.

In one embodiment, the metal coordinating moiety corresponds to Formula (2):

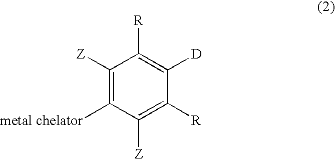

wherein

each Z is independently hydrogen, hydroxy, or thiol provided, however, that at least one Z substituent is other than hydrogen;

each R is independently bromo or iodo; and

D is hydrogen or a substituent selected to influence stability, biodistribution and/or toxicity.

In general, the metal chelator may be acyclic or cyclic. For example, metal chelators include polycarboxylic acids such as EDTA, DTPA, DCTA, DOTA, TETA, or analogs or homologs thereof. To provide greater stability under physiological conditions, however, macrocyclics, e.g., triaza and tetraza macrocycles, are generally preferred. In some embodiments, the macrocyclic metal chelator is cyclen or tacn.

In one embodiment, the metal coordinating moiety comprises a substituted heterocyclic ring where the heteroatom is nitrogen. Typically, the heterocyclic ring comprises from about 9 to about 15 atoms, at least 3 of these ring atoms being nitrogen. In one example of this embodiment, the heterocyclic ring comprises 3-5 ring nitrogen atoms where at least one of the ring nitrogen atoms is substituted. For these embodiments, the ring carbon atoms are optionally substituted. One such macrocycle corresponds to Formula (3):

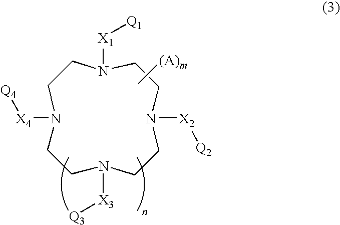

wherein

n is 0, 1 or 2;

m is 0-20 wherein when m is greater than 0, each A is independently C₁₋₂₀ alkyl or aryl optionally substituted by one or more aryl, C₁₋₂₀ alkyl, carbaldehyde, keto (—C(O)), carboxyl (—CO₂H), cyano (—CN), halo, nitro (—NO₂), amido (—C(O)NH—), polypeptides (e.g., polyserine), sulfato (—OSO₃H), sulfito (—SO₃H), phosphato (—OPO₃H₂), phosphito (—PO₃H₂), hydroxyl, oxy, ether, polyether (e.g., polyethylene glycols), C₄₋₂₀ carbohydrate, mercapto (—SH) or thio;

X₁, X₂, X₃ and X₄ are independently optionally substituted methylene where the substituents are selected from the group consisting of aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto and thio;

Q₁ is

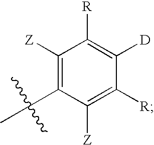

Q₂, Q₃ and Q₄ are independently selected from the group consisting of optionally substituted methylthio, carboxyl, phosphonate, sulfonate, and

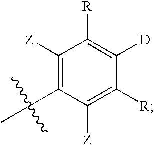

each Z is independently hydrogen, hydroxy, or thiol provided, however, that at least one Z substituent is other than hydrogen;

each R is independently bromo or iodo; and

D is selected from the group consisting of hydrogen, fluoro, chloro, bromo, iodo, carboxyl, cyano, nitro, amido, polypeptides, hydroxyl, amino, ammonium, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (3), D is hydrogen, bromo, iodo, carboxyl, or hydroxyl.

Typically, when Q₂-Q₄ are substituted, the substituents are selected from the group consisting of fluoro, chloro, bromo, iodo, carboxyl, cyano, nitro, amido, polypeptides, hydroxyl, amino, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (3), X₁-X₄ are independently methylene optionally substituted by C₁₋₆ alkyl, halo, or hydroxyl.

When the metal coordinating moiety corresponds to Formula (3) and m is greater than zero, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto or thio substituents. In addition, when A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, polypeptides, sulfato, sulfito, phosphate, phosphito, hydroxyl, oxy, mercapto and thio.

Further, for the metal coordinating moieties of Formula (3), the A substituent, if present, is bonded to any of the ring carbon atoms. Further, each ring carbon atom may be substituted by one or two A substituents so that the number of possible A substituents varies with the number of ring carbon atoms. In one embodiment of metal coordinating moieties of Formula (3) having at least one A substituent, each A is independently aryl or C₁₋₈ alkyl optionally substituted with one or more aryl, keto, carboxyl, cyano, nitro, C₁₋₂₀ alkyl, amido, polypeptides, sulfato, sulfito, phosphate, phosphito, oxy and thio. For example, each A may be aryl or C₁₋₆ alkyl optionally substituted with one or more aryl, keto, amido, polypeptides and oxy. By way of further example, each A may be methyl.

In general, as the value of n increases, the size of the macrocycle increases. In this manner, the size of the macrocycle may be controlled to match the size and coordination capacity of the metal to be coordinated.

Exemplary metal coordinating moieties of Formula (3) include:

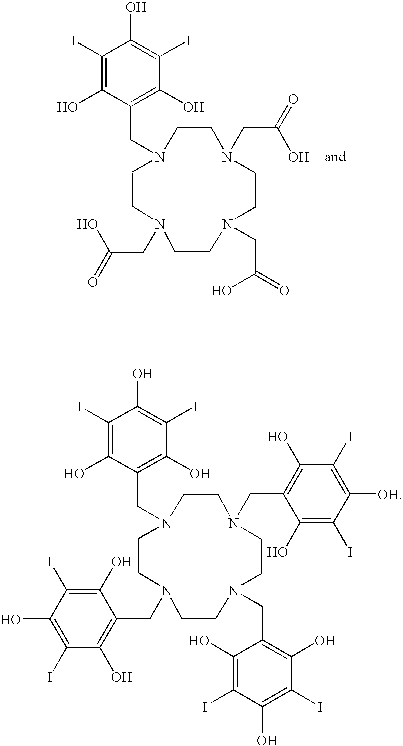

Alternatively, the metal coordinating moieties may comprise a substituted chain of carbon and nitrogen atoms instead of a heterocyclic ring. As used herein the chain of nitrogen and carbon may be referred to as the “backbone” or the “chain of atoms”. Typically, the chain of atoms comprises from about 4 to about 10 atoms, at least 2 of said atoms being nitrogen. Preferably, the chain of atoms comprises 2-4 nitrogen atoms wherein at least one of the chain nitrogen atoms is substituted. The backbone carbon atoms are optionally substituted. Typically, the backbone nitrogen atoms are separated from each other by two carbon atoms. In this embodiment, the metal coordinating moiety typically has the following Formula (4):

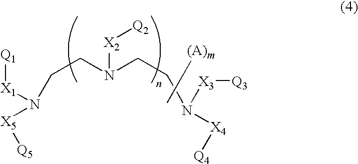

wherein

n is 0, 1 or 2;

m is 0-12 wherein when m is greater than 0, each A is independently C₁₋₂₀ alkyl or aryl optionally substituted by one or more aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto or thio;

X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₁₋₂₀ carbohydrate, mercapto and thio;

### Q₁ is

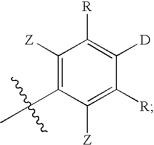

Q₂, Q₃, Q₄ and Q₅ are independently selected from the group consisting of optionally substituted methylthio, carboxyl, phosphonate, sulfonate, and

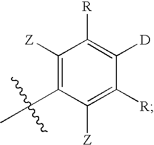

each Z is independently hydrogen, hydroxy, or thiol provided, however, that at least one Z substituent is other than hydrogen;

each R is independently bromo or iodo; and

D is selected from the group consisting of hydrogen, fluoro, chloro, bromo, iodo, carboxyl, cyano, nitro, amido, polypeptides, hydroxyl, amino, ammonium, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (4), D is hydrogen, bromo, iodo, carboxyl, or hydroxyl.

Typically, when Q₂-Q₅ are substituted, the substituents are selected from the group consisting of fluoro, chloro, bromo, iodo, carboxyl, cyano, nitro, amido, polypeptides, hydroxyl, amino, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (4), X₁-X₅ are independently methylene optionally substituted by C₁₋₆ alkyl, halo, or hydroxyl.

When the metal coordinating moiety corresponds to Formula (4) and m is greater than zero, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto or thio substituents. In addition, when A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, polypeptides, sulfato, sulfito, phosphato, phosphito, hydroxyl, oxy, mercapto and thio.

Further, for the metal coordinating moieties of Formula (4), the A substituent, if present, is bonded to any of the ring carbon atoms. Further, each ring carbon atom may be substituted by one or two A substituents so that the number of possible A substituents varies with the number of ring carbon atoms. In one embodiment of metal coordinating moieties of Formula (4) having at least one A substituent, each A is independently aryl or C₁₋₈ alkyl optionally substituted with one or more aryl, keto, carboxyl, cyano, nitro, C₁₋₂₀ alkyl, amido, polypeptides, sulfato, sulfito, phosphate, phosphito, oxy and thio. For example, each A may be aryl or C₁₋₆ alkyl optionally substituted with one or more aryl, keto, amido, polypeptides and oxy. By way of further example, each A may be methyl.

In general, as the value of n increases, the length of the chain of atoms increases. In this manner, the length of the backbone may be controlled to match the size and coordination capacity of the metal to be coordinated.

Exemplary metal coordinating moieties of Formula (4) include:

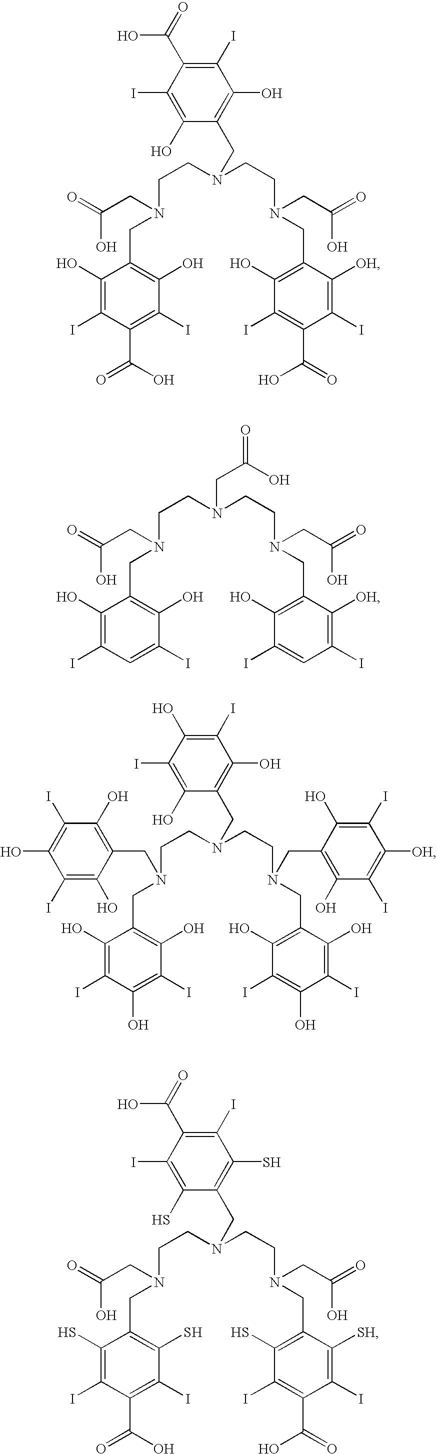

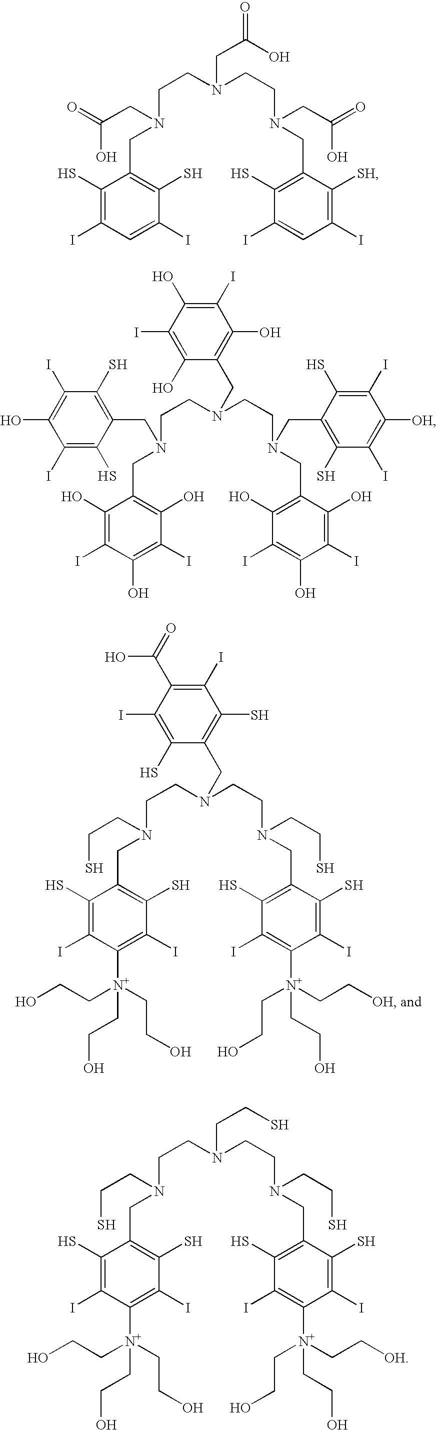

For any of the above embodiments, the metal coordinating moiety may be complexed with a metal, M, thereby forming a metal complex.

In one embodiment where the metal coordinating moiety is a heterocyclic ring and complexed with a metal, M, the complex has the following Formula (5):

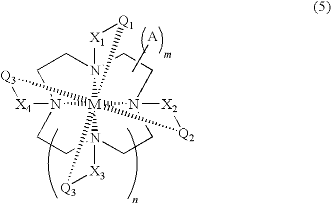

wherein

n is 0, 1 or 2;

m is 0-20 wherein when m is greater than 0, each A is independently C₁₋₂₀ alkyl or aryl optionally substituted by one or more aryl, C₁₋₂₀ alkyl, carbaldehyde, keto, carboxyl, cyano, halo, nitro, amido, polypeptides, sulfato, sulfito, phosphate, phosphito, hydroxyl, oxy, ether, polyether, C₄₋₂₀ carbohydrate, mercapto or thio;

X₁, X₂, X₃ and X₄ are independently optionally substituted methylene where the substituents are selected from the group consisting of aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto and thio;

Q₁ is

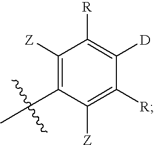

Q₂, Q₃ and Q₄ are independently selected from the group consisting of optionally substituted methylthio, carboxyl, phosphonate, sulfonate, and

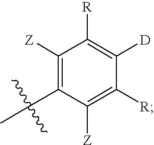

each Z is independently hydrogen, hydroxy, or thiol provided, however, that at least one Z substituent is other than hydrogen;

each R is independently bromo or iodo;

D is selected from the group consisting of hydrogen, fluoro, chloro, bromo, iodo, carboxyl, cyano, nitro, amido, polypeptides, hydroxyl, amino, ammonium, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito; and

M is selected from the group consisting of W, Hg, Pb, Lu, Lu-177, Y, Y-90, In, In-111, Tc, Tc═O, Tc-99m, Tc-99 m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

In one embodiment, for metal coordinating moieties of Formula (5), D is hydrogen, bromo, iodo, carboxyl, or hydroxyl.

Typically, when Q₂-Q₄ are substituted, the substituents are selected from the group consisting of fluoro, chloro, bromo, iodo, carboxyl, cyano, nitro, amido, polypeptides, hydroxyl, amino, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (5), X₁-X₄ are independently methylene optionally substituted by C₁₋₆ alkyl, halo, or hydroxyl.

While not depicted in Formula (5), the hydroxyl or thiol group(s) of the halogen-substituted (thio)phenol or ((di)thio)resorcinol derivative may independently participate in the coordination of the metal. Accordingly, in some embodiments, no hydroxyl or thiol group(s) directly participate in the coordination of the metal, while in other embodiments one or two of the hydroxyl or thiol group(s) participate in the coordination of the metal. Both the nature of the metal selected and the particular metal coordinating moiety selected will determine whether the hydroxyl or thiol group(s) of the halogen-substituted (thio)phenol or ((di)thio)resorcinol derivative participate in the coordination of the metal. Further, when the metal coordinating moiety comprises a resorcinol derivative, an oxygen atom from each resorcinol may be involved in the bonding of the metal at one time or another due to the equilibrium present. Both hydroxyl oxygens from a single resorcinol moiety, however, may not bond to the metal at the same time.

Exemplary metal coordinating complexes of Formula (5) include:

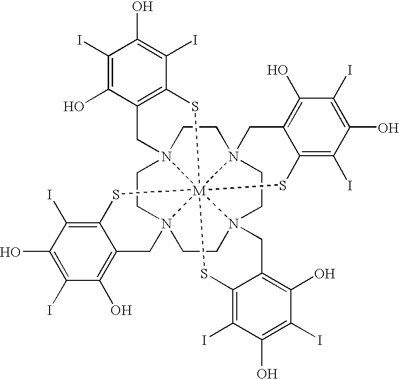

wherein M is Pb or Bi;

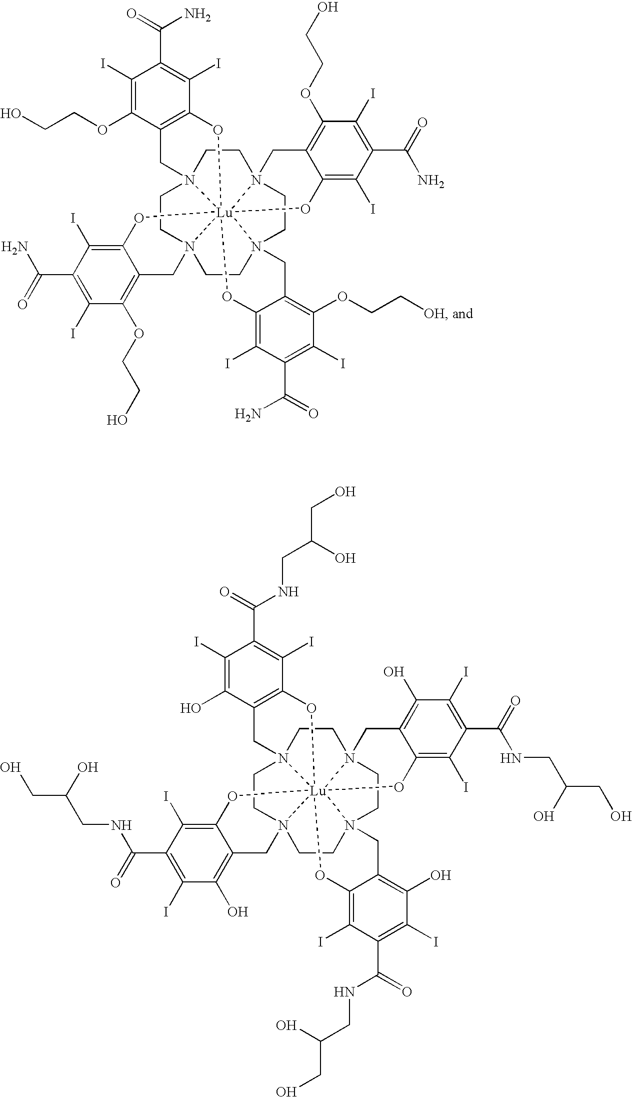

Alternatively, in one embodiment where the metal coordinating moiety comprises a chain of atoms and is complexed with a metal, M, the complex has the following Formula (6):

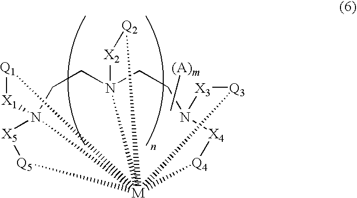

wherein

n is 0, 1 or 2;

m is 0-12 wherein when m is greater than 0, each A is independently C₁₋₂₀ alkyl or aryl optionally substituted by one or more aryl, C₁₋₂₀ alkyl, carbaldehyde, keto, carboxyl, cyano, halo, nitro, amido, polypeptides, sulfato, sulfito, phosphate, phosphito, hydroxyl, oxy, ether, polyether, C₄₋₂₀ carbohydrate, mercapto or thio;

X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto and thio;

Q₁ is

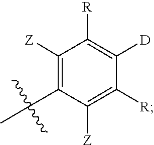

Q₂, Q₃, Q₄ and Q₅ are independently selected from the group consisting of optionally substituted methylthio, carboxyl, phosphonate, sulfonate, and

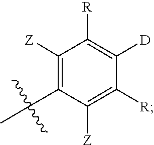

each Z is independently hydrogen, hydroxy, or thiol provided, however, that at least one Z substituent is other than hydrogen;

each R is independently bromo or iodo;

D is selected from the group consisting of hydrogen, fluoro, chloro, bromo, iodo, carboxyl, cyano, nitro, amido, polypeptides, hydroxyl, amino, ammonium, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito; and

M is selected from the group consisting of W, Hg, Pb, Lu, Lu-177, Y, Y-90, In, In-111, Tc, Tc═O, Tc-99m, Tc-99 m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

In one embodiment, for metal coordinating moieties of Formula (6), D is hydrogen, bromo, iodo, carboxyl, or hydroxyl.

Typically, when Q₂-Q₅ are substituted, the substituents are selected from the group consisting of fluoro, chloro, bromo, iodo, carboxyl, cyano, nitro, amido, polypeptides, hydroxyl, amino, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, polypeptides, hydroxyl, amino, sulfato, sulfito, phosphato, and phosphito.

In one embodiment, for metal coordinating moieties of Formula (6), X₁-X₅ are independently methylene optionally substituted by C₁₋₆ alkyl, halo, or hydroxyl.

Exemplary metal coordinating complexes of Formula (6) include:

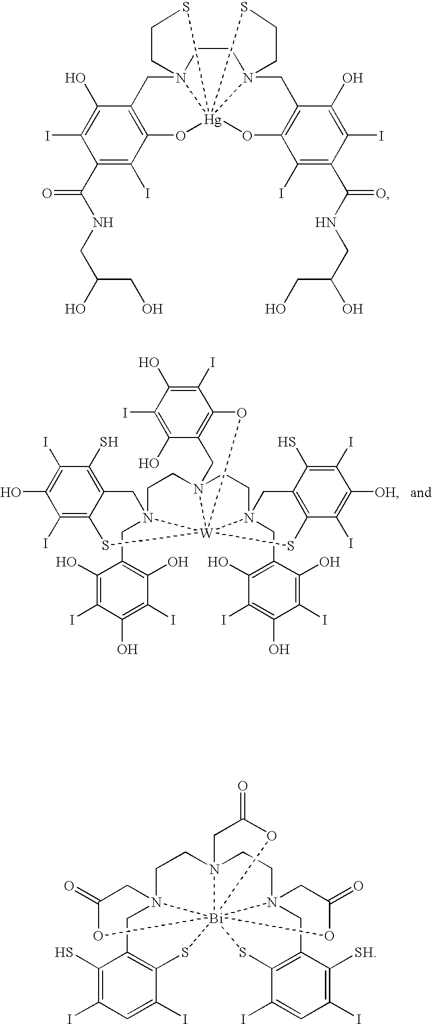

While not depicted in Formula (6), the hydroxyl or thiol group(s) of the halogen-substituted (thio)phenol or ((di)thio)resorcinol derivative may independently participate in the coordination of the metal. Accordingly, in some embodiments, no hydroxyl or thiol group(s) directly participate in the coordination of the metal, while in other embodiments one or two of the hydroxyl or thiol group(s) participate in the coordination of the metal. In one embodiment, both groups participate at one time or another, as illustrated in the following representation:

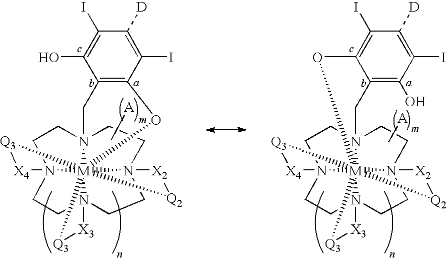

where the two oxygen atoms are interconverting due to breaking and reformation of M-O (letters a, b, and c are recited to better show the interconversion between the two oxygen atoms). The above representation is illustrative only.

Both the nature of the metal selected and the particular metal coordinating moiety selected will determine whether the hydroxyl or thiol group(s) of the (thio)phenol or ((di)thio)resorcinol derivative participate in the coordination of the metal. Further, when the metal coordinating moiety comprises a resorcinol derivative, both of the oxygen atoms are involved in the bonding of the metal at one time or another due to the equilibrium present. Both hydroxyl oxygens, however, are not bond to the same metal at the same time. Similarly, certain metals, e.g., lead and bismuth, are effectively coordinated by thiol groups. In the case of a dithioresorcinol derivate, although both of the thiol sulfur atoms may be involved in the binding of the metal at one time or another, they are not bond to the same metal at the same time.

Whether the preferred complex corresponds to Formula (5) or Formula (6) typically depends on the particular metal selected for coordination. For example, for yttrium and lanthanides, the complex corresponding to Formula (5) is preferred. Formula (5) is also preferred for iron, copper, and manganese while Formula (6) is the preferred complex for the remaining transition metals. The preferred complex for any particular metal is related to the potential for transmetallation with endogenous ion. Thus, Formula (5) provides greater stability with high exchange metals, including, but not limited to, yttrium, lanthanides, and gallium. Transmetallation with endogenous ions does not present as great a concern for regular transition metals.

Macrocyclic metal coordinating moieties with three-dimensional cavities often form metal complexes with high stability. These complexes often exhibit selectivity for certain metal atoms based on metal size and coordination chemistry, and capability to adopt a preorganized conformation in the uncomplexed form, which facilitates metal complexation. The selection of appropriate macrocyclic metal coordinating moieties and metals is known by those skilled in the art.

In addition, the preferred value of n, and hence the size or length of the metal coordinating moiety, depends upon the particular metal to be coordinated. For yttrium and lanthanides, for example, n is preferably 1. For transition metals, n is typically 0 or 1. For manganese, technetium, lead, and bismuth, n is 0, 1, or 2 depending on the value of X₁-X₄, which is selected to provide the maximum complex stability.

### General Synthesis

Several generic synthetic schemes for the preparation of halo-substituted (thio)phenol or ((di)thio)resorcinol-bearing metal coordinating moieties are shown below.

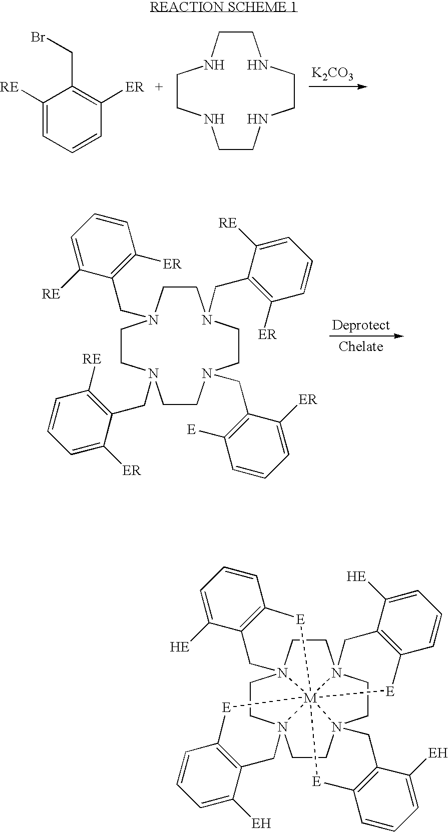

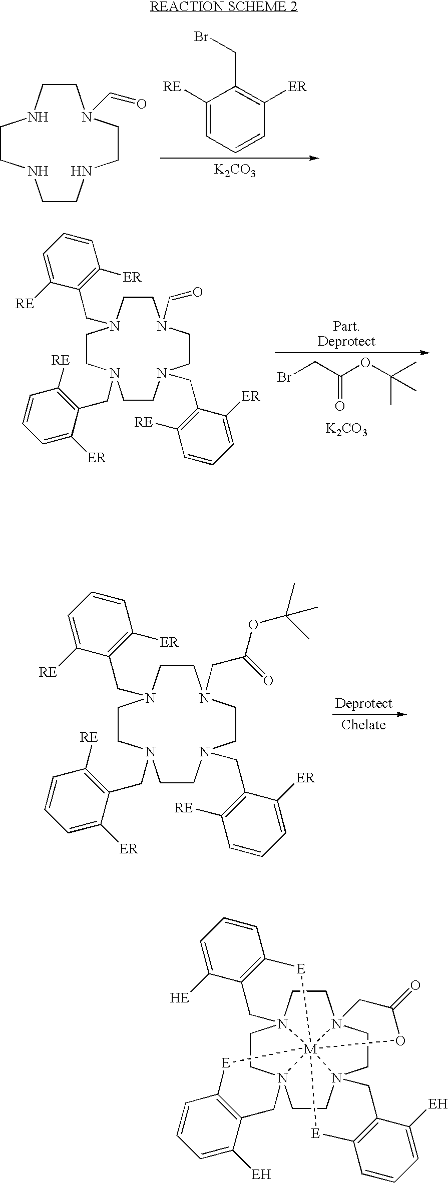

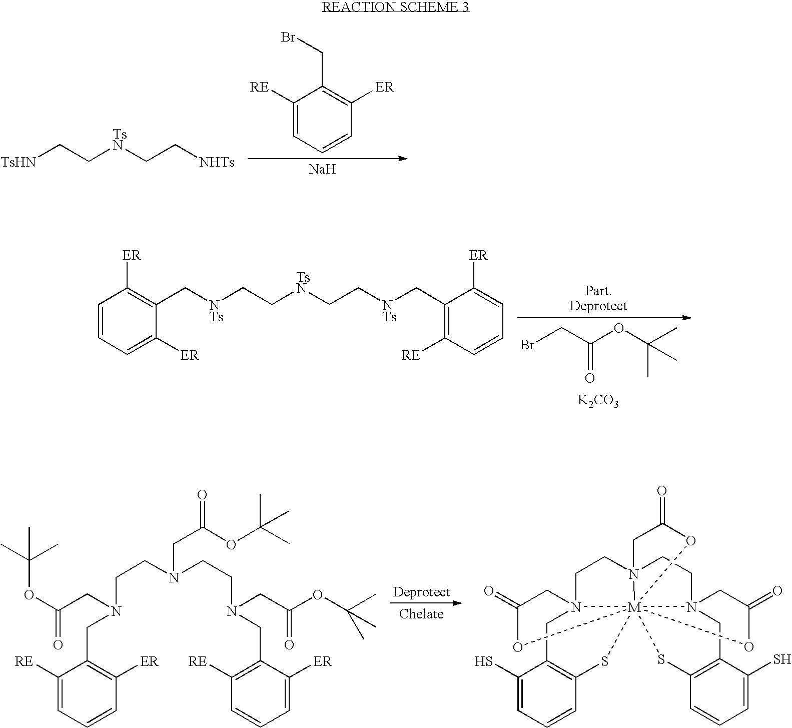

In the reaction schemes above, E is oxygen or sulfur; R is a protecting group (e.g., Bz, t-Bu, SiMe₃, or SiPr₃); and M is a metal of radiological importance (e.g., Pb, Bi, Lu, Gd, In, Ga, Hg, or W).

Synthesis of an exemplary tetra-resorcinol metal coordinating moiety and corresponding complex can be performed as follows:

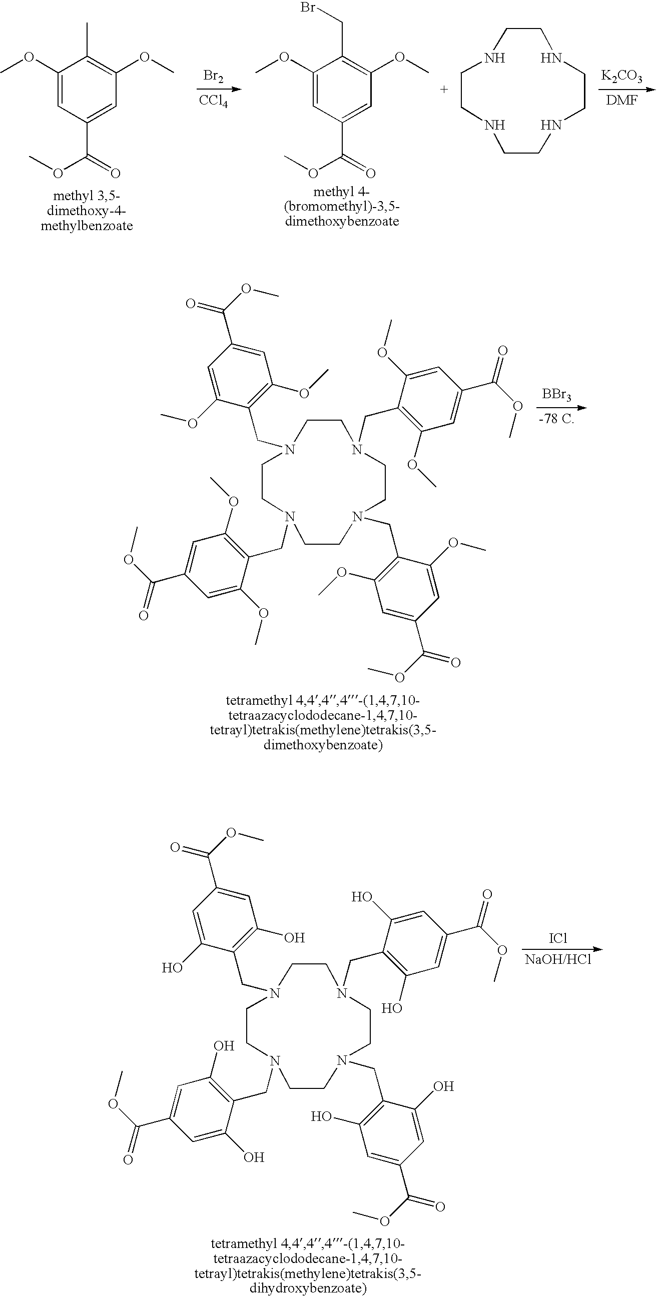

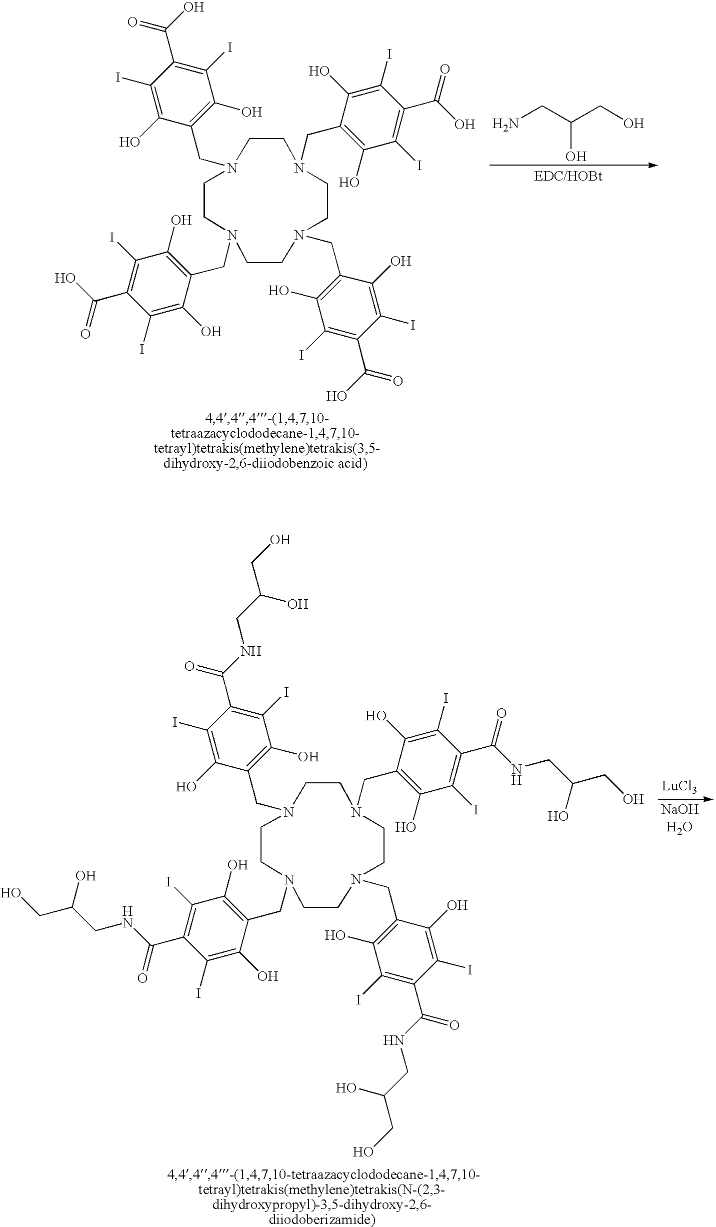

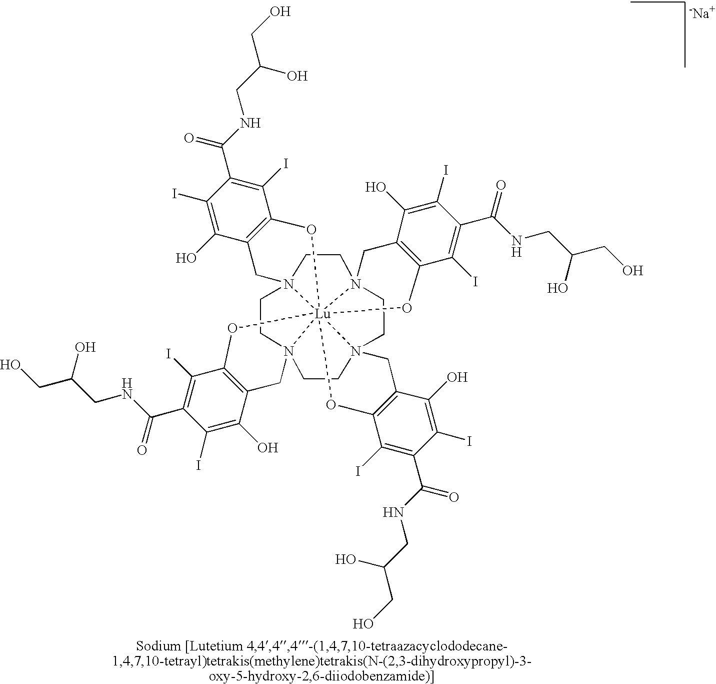

### Methyl 4-(bromomethyl)-3,5-dimethoxybenzoate

A carbon tetrachloride solution of methyl 3,5-dimethoxy-4-benzoate is treated with 1.1 equivalents of bromine at room temperature. A high intensity lamp may be required to complete the bromination. The reaction is treated saturated aqueous sodium bicarbonate and the organic extract dried with magnesium sulfate. The product is isolated by evaporation of the solvent and may need to be purified by crystallization or chromatography.

### Tetramethyl 4,4′,4″,4′″-(1,4,7,10-tetraazacyclododecane-1,4,7,10-tetrayl)tetrakis-(methylene)tetrakis(3,5-dimethoxybenzoate)

Cyclen may be stirred with 4.4 equivalents potassium carbonate in dry dimethylformamide under inert atmosphere. Methyl 4-(bromomethyl)-3,5-dimethoxybenzoate will alkylate the cyclen, with heat if needed. The product could be isolated by crystallization from a suitable solvent such as acetonitrile.

### Tetramethyl 4,4′,4″,4′″-(1,4,7,10-tetraazacyclododecane-1,4,7,10-tetrayl)tetrakis-(methylene)tetrakis(3,5-dihydroxybenzoate)

The resorcinol moieties may be unmasked by treatment of tetramethyl 4,4′,4″,4′″-(1,4,7,10-tetraazacyclododecane-1,4,7,10-tetrayl)tetrakis-(methylene)tetrakis(3,5-dimethoxybenzoate), in dry dichloromethane at −78° C., with 12 equivalents of boron tribromide. After stirring at −78° C. for 30 minutes, the reaction would be allowed to stir at 0° C. for an additional hour. After concentrating the mixture, the product might be purified via chromatography.

### 4,4′,4″,4′″-(1,4,7,10-Tetraazacyclododecane-1,4,7,10-tetrayl)tetrakis(methylene)-tetrakis(3,5-dihydroxy-2,6-diiodobenzoic acid)

Tetramethyl 4,4′,4″,4′″-(1,4,7,10-tetraazacyclododecane-1,4,7,10-tetrayl)tetrakis-(methylene)tetrakis(3,5-dihydroxybenzoate) stirring in water-acetonitrile at room temperature, could be treated with a solution of ICI, 9 equivalents in 37% HCl. The reaction mixture would be allowed to stir for several days, while the reaction is monitored by HPLC for completeness. Portions of methanol may need to be added from time to time in order to maintain a clear solution. The ester intermediate could be isolated by precipitation by the addition of water. Saponification of the esters would be accomplished by treatment with sodium hydroxide in aqueous methanol, followed by acidification, to give the desired carboxylic acid.

### 4,4′,4″,4′″-(1,4,7,10-Tetraazacyclododecane-1,4,7,10-tetrayl)tetrakis(methylene)-tetrakis(N-(2,3-dihydroxypropyl)-3,5-dihydroxy-2,6-diiodobenzamide)

4,4′,4″,4′″-(1,4,7,10-Tetraazacyclododecane-1,4,7,10-tetrayl)tetrakis(methylene)-tetrakis(3,5-dihydroxy-2,6-diiodobenzoic acid), dissolved in water-acetonitrile, could be coupled to aminopropanediol by treatment with N-(3-dimethylaminopropyl)-N′-ethylcarbodiimide (EDC) in the presence of 1-hydroxybenzotriazole (HOBt). The product would be isolated in its pure form after reverse phase chromatography.

### Sodium[lutetium 4,4′,4″,4′″-(1,4,7,10-tetraazacyclododecane-1,4,7,10-tetrayl)tetrakis(methylene)tetrakis(N-(2,3-dihydroxypropyl)-3-oxy-5-hydroxy-2,6-diiodobenzamide)]

Formation of the lutetium complex would be done in water with heating. The pH of the reaction mixture could be adjusted with a base such as sodium hydroxide to allow isolation of the lutetium complex as the monosodium salt. Additional purification could be accomplished by reverse phase chromatography.

Synthesis of an exemplary tri-resorcinol metal coordinating moiety and corresponding complex can be performed as follows:

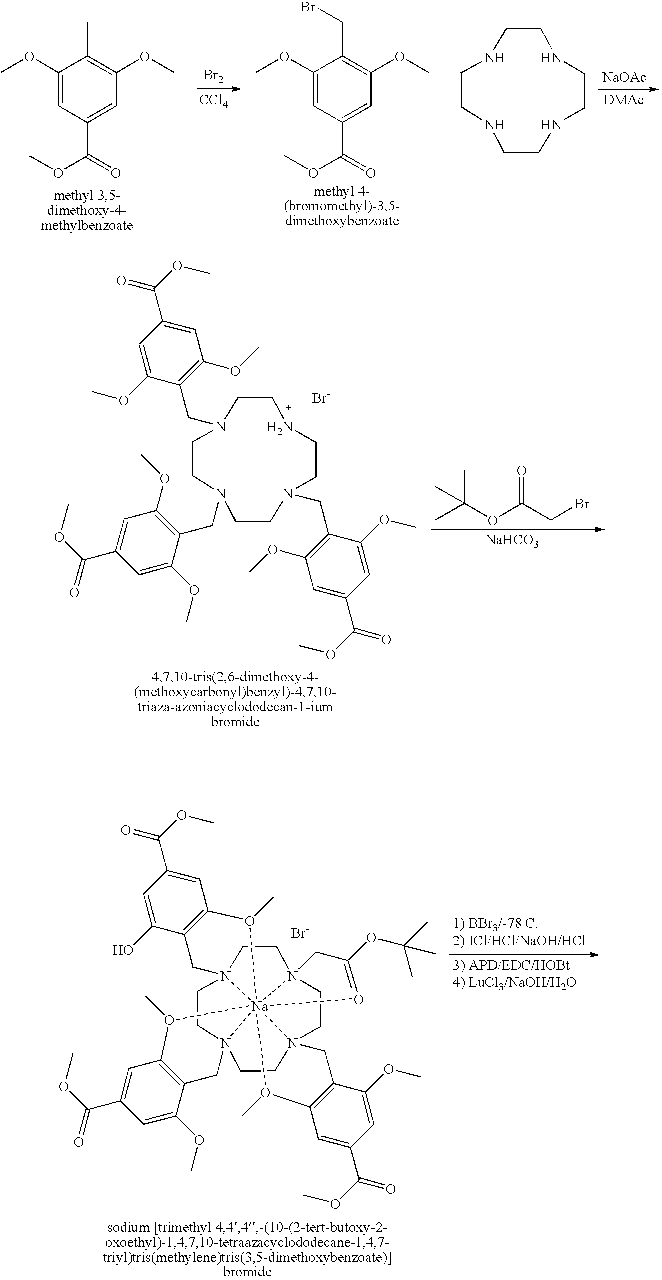

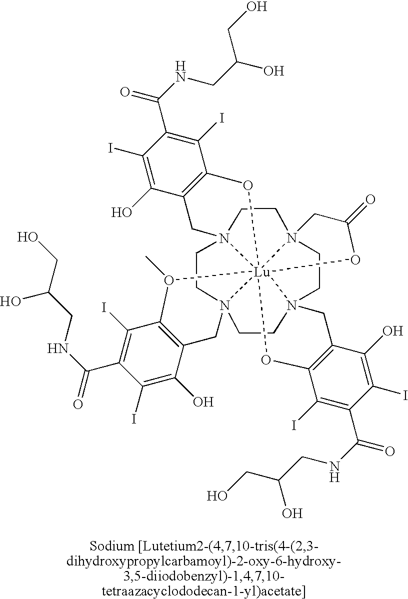

### 4,7,10-Tris(2,6-dimethoxy-4-(methoxycarbonyl)benzyl)-4,7,10-triaza-azoniacyclododecan-1-ium bromide

Cyclen may be trialkylated using only 3.3 equivalents of methyl 4-(bromomethyl)-3,5-dimethoxybenzoate and 3.3 equivalents sodium acetate in dimethylacetamide. The product may be isolated as the monohydrobromide salt by crystallization.

### Sodium [trimethyl 4,4′,4″-(10-(2-tert-butoxy-2-oxoethyl)-1,4,7,10-tetraazacyclodo-decane-1,4,7-triyl)tris(methylene)tris(3,5-dimethoxybenzoate)] bromide

The above HBr salt may be free-based using aqueous sodium hydroxide and ether or other suitable organic extractant. Treatment of the free base in acetonitrile with sodium bicarbonate and one equivalent tert-butyl bromoacetate may give the sodium complex as the bromide salt.

### Sodium [lutetium 2-(4,7,10-tris(4-(2,3-dihydroxypropylcarbamoyl)-2-oxy-6-hydroxy-3,5-diiodobenzyl)-1,4,7,10-tetraazacyclododecan-1-yl)acetate]

Analogous, serial, treatment of the sodium complex to deprotect the ether moieties, followed by treatment with ICI, saponification, and conjugation to aminopropanediol should give rise to the desired chelate. Complexation with lutetium would be carried out in the usual way.

Synthesis of an exemplary acyclic di-resorcinol metal coordinating moiety and corresponding complex can be performed as follows:

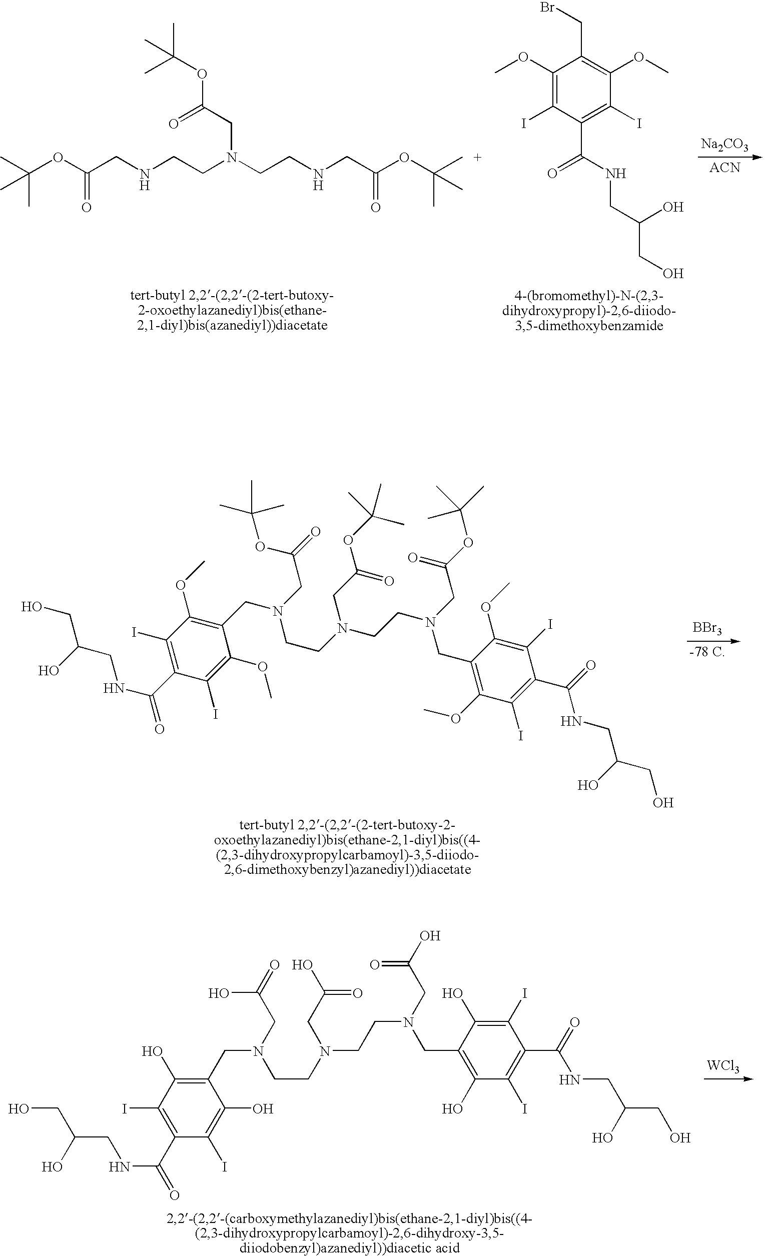

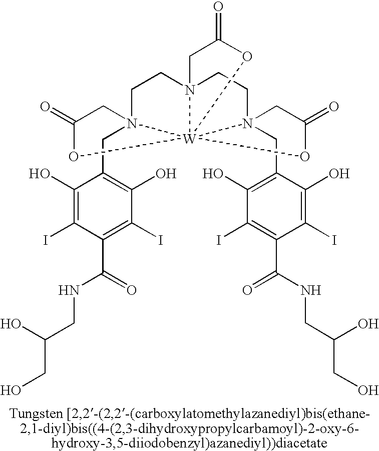

### tert-Butyl 2,2′-(2,2′-(2-tert-butoxy-2-oxoethylazanediyl)bis(ethane-2,1-diyl)bis((4-(2,3-dihydroxypropylcarbamoyl)-3,5-diiodo-2,6-dimethoxybenzyl)azanediyl))-diacetate

tert-Butyl 2,2′-(2,2′-(2-tert-butoxy-2-oxoethylazanediyl)bis(ethane-2,1-diyl)bis(azanedi-yl))diacetate may be dialkylated using 4-(bromomethyl)-N-(2,3-dihydroxypropyl)-2,6-diiodo-3,5-dimethoxybenzamide and sodium carbonate in acetonitrile.

### 2,2′-(2,2′-(Carboxymethylazanediyl)bis(ethane-2,1-diyl)bis((4-(2,3-dihydroxypropylcarbamoyl)-2,6-dihydroxy-3,5-diiodobenzyl)azanediyl))diacetic acid

In a fashion already described, the ester/ether may be concomitantly deprotected by treatment with BBr3 in dry dichloromethane at −78 C. The resulting resorcinol-carboxylic acid may be purified using reverse phase chromatography.

### Tungsten [2,2′-(2,2′-(carboxylatomethylazanediyl)bis(ethane-2,1-diyl)bis((4-(2,3-dihydroxypropylcarbamoyl)-2-oxy-6-hydroxy-3,5-diiodobenzyl)azanediyl))diacetate

Finally, the tungsten complex may be prepared in water. Modification of pH may be required, followed by purification using reverse phase chromatography, to isolate the desired complex.

### Metallopharmaceutical Compositions

Metallopharmaceutical compositions of the present invention comprise a metal coordinating moiety, complexed to a metal, dispersed in a pharmaceutically acceptable radiological carrier. The pharmaceutically acceptable carrier, also known in the art as an excipient, vehicle, auxiliary, adjuvant, or diluent, is typically a substance which is pharmaceutically inert, confers a suitable consistency or form to the composition, and does not diminish the therapeutic or diagnostic efficacy of the conjugate. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier will also, in part, be a function of the route of administration. In general, the metallopharmaceutical compositions of the invention can be formulated with conventional pharmaceutically acceptable carriers for any route of administration so long as the target tissue is available via that route. For example, suitable routes of administration include, but are not limited to, oral, parenteral (e.g., intravenous, intraarterial, subcutaneous, subcutaneous, intramuscular, intracapsular, intraspinal, or intraperitoneal), intravesical, intrathecal, enteral, pulmonary, intralymphatic, intracavital, transurethral, intradermal, intramammary, buccal, orthotopic, intralesional, percutaneous, endoscopical, transmucosal, and intestinal administration.

Pharmaceutically acceptable carriers for use in the compositions of the present invention are well known to those of ordinary skill in the art and are selected based upon a number of factors: the particular complex used, and its concentration, stability and intended bioavailability; the disease, disorder or condition being diagnosed with the composition; the subject, its age, size and general condition; and the route of administration. Suitable pharmaceutically acceptable carriers include those that are suitable for injection such as aqueous buffer solutions; e.g., tris(hydroxymethyl)amino methane (and its salts), phosphate, citrate, bicarbonate, etc., sterile water for injection, physiological saline, and balanced ionic solutions containing chloride and/or bicarbonate salts of normal blood plasma cations such as Ca, Na, K and Mg, and other halides, carbonates, sulphates, phosphates of Na, K, Mg, Ca. Other buffer solutions are described in Remington's Practice of Pharmacy, Eleventh Edition, for example on page 170. The vehicles may advantageously contain a small amount (e.g., from about 0.01 to about 15.0 mole %) of a chelating agent such as ethylenediamine tetraacetic acid (EDTA), calcium disodium EDTA, or other pharmaceutically acceptable chelating agents such as calcium monosodium DTPA-BMEA (Versetamide; Mallinckrodt Inc.). The composition may further comprise non-radiographic additives selected from the group consisting of excipients, such as, for example, glycerol, polyethylene glycol or dextran, and anticlotting agents, such as, for example, heparin or hirudin.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, JOURNAL OF PHARMACEUTICAL SCIENCES, Vol. 52, No. 10, pp. 917-927 (1963).

### Dosage

The diagnostic compositions are administered in doses effective to achieve the desired enhancement of the image. The dosages can be readily determined by those with ordinary skill in diagnosing disease. Such doses may vary widely, depending upon the particular metal coordinating moiety selected, the organs or tissues which are the subject of the imaging procedure, the imaging procedure, the imaging equipment being used, and the like. Generally, the solution is formulated at varying concentrations of the X-ray opaque substance. These different products are used for different indications and patient conditions. In one embodiment, depending on the particular product and concentration, osmolalities range from about 290 to about 2400 mOsm/kg water.

In general, parenteral dosages will range from about 0.001 to about 1.0 mMol of metal coordinating moiety complex per kg of patient body weight. Preferred parenteral dosages generally range from about 0.01 to about 0.5 mMol of metal ion complex per kg of patient body weight. Enteral dosages generally range from about 0.5 to about 100 mMol, preferably from about 1.0 to about 10 mMol of metal ion complex per kg of patient body weight.

Further, in forming diagnostic radioactive complexes, it is generally preferred to form radioactive complexes in solutions containing radioactivity at concentrations of from about 0.01 millicurie (mCi) to 100 mCi per mL. Generally, the unit dose to be administered has a radioactivity of about 0.01 mCi to about 100 mCi, preferably about 1 mCi to about 30 mCi. The solution to be injected at unit dosage is from about 0.01 mL to about 10 mL. The amount of radiolabeled complex appropriate for administration is dependent upon the distribution profile of the chosen complex in the sense that a rapidly cleared complex may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at the non-target tissue.

### DEFINITIONS

The compounds described herein may have asymmetric centers. Compounds of the present invention containing an asymmetrically substituted atom may be isolated in optically active or racemic form. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The present invention includes all isotopes of atoms occurring in the present compounds. Isotopes include those atoms having the same atomic number but different mass numbers.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “amido” as used herein includes substituted amido moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, sulfato, sulfito, phosphate, phosphito, hydroxyl, oxy, mercapto, and thio substituents.

The term “amino” as used herein includes substituted amino moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, sulfato, sulfito, phosphate, phosphito, hydroxyl, oxy, mercapto, and thio substituents.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “carbaldehyde” as used herein denotes an aldehyde functional group (CHO) attached to a ring (e.g., C₆H₁₁CHO is referred to as cyclohexanecarbaldehyde).

The terms “complex”, “metal complex”, and “metal coordinating complex” refer to a metal coordinating moiety of the invention, e.g. Formula (2), complexed or coordinated with a metal.

The terms “halogen” or “halo” as used herein alone or as part of another group refer to chlorine, bromine, fluorine, and iodine.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring. The heterocyclo group preferably has 1 to 5 nitrogen atoms in the ring, and may be bonded to the remainder of the molecule through a carbon atom. Exemplary heterocyclics include macrocyclics, cyclen, DOTA, DOTMA, DOTP, and TETA.

The “heterosubstituted alkyl” moieties described herein are alkyl groups in which a carbon atom is covalently bonded to at least one heteroatom and optionally with hydrogen, the heteroatom being, for example, a nitrogen atom.

The term “metallopharmaceutical” as used herein refers to a pharmaceutically acceptable compound comprising a metal, wherein the compound is useful for imaging or treatment.

The term “peptide” as used herein denotes any of various natural or synthetic compounds containing two or more amino acids linked by the carboxyl group of one amino acid and the amino group of another. Generally, “polypeptides” comprise between 10 and 100 amino acids.

As used herein, a “phenol derivative” comprises a hydroxyphenyl moiety.

As used herein, a “thiophenol derivative” comprises a thiophenyl moiety.

As used herein, a “resorcinol derivative” comprises a m-dihydroxybenzene moiety.

As used herein, a “thioresorcinol derivative” comprises a resorcinol derivative wherein one of the hydroxyl functional groups has been replaced by a thiol functional group.

As used herein, a “dithioresorcinol derivative” comprises a resorcinol derivative wherein both of the hydroxyl functional groups have been replaced by thiol functional groups.

The following example is prophetic.

### Example 1

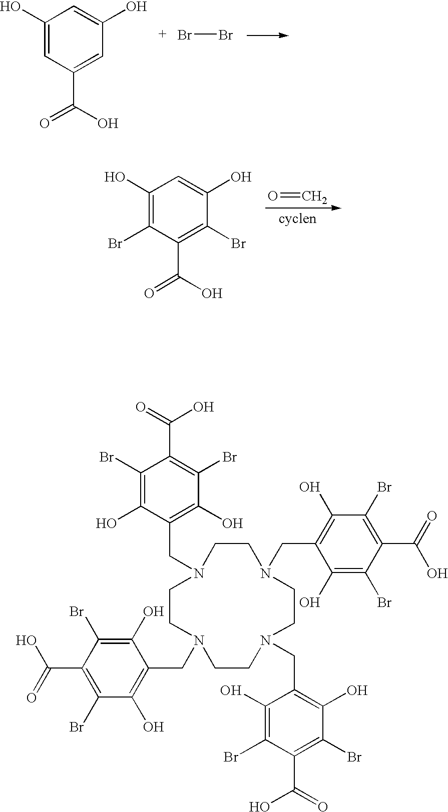

1. A metal coordinating moiety comprising a metal chelator and a halogen-substituted phenol, thiophenol, resorcinol, thioresorcinol, or dithioresorcinol derivative, wherein the metal coordinating moiety is represented by the formula: 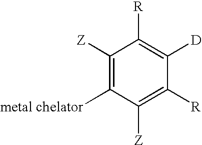
wherein:
each Z is independently hydrogen, hydroxy, or thiol; provided, that at least one Z substituent is other than hydrogen;
each R is independently bromo or iodo; and
D is selected from fluoro, chloro, bromo, iodo, carboxyl, cyano, nitro, amido, hydroxyl, amino, ammonium, sulfato, sulfito, phosphato, phosphito, ether, polyether, aryl, or C₁₋₂₀ alkyl optionally substituted with one or more of C₁₋₂₀ alkyl, carboxyl, cyano, nitro, amido, hydroxyl, amino, sulfato, sulfito phosphato, and phosphito.

2. The metal coordinating moiety of claim 1 wherein the metal chelator comprises a polycarboxylic acid, a triaza- or tetraza-macrocycle, or a substituted heterocyclic ring.

3. The metal coordinating moiety of claim 2 wherein the metal chelator comprises a polycarboxylic acid selected from EDTA, DTPA, DCTA, DOTA, TETA, or analogs or homologs thereof.

4. The metal coordinating moiety of claim 1 wherein the metal coordinating moiety is complexed with a metal, the metal selected from a radioisotope, paramagnetic metal, or x-ray opaque metal.

5. The metal coordinating moiety of claim 4 wherein the metal is selected from W, Hg, Pb, Lu, Lu-177, Y, Y-90, In, In-111, Tc, Tc═O, Tc-99m, Tc-99m-O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi Bi-212, As or As-211.

6. The metal coordinating moiety of claim 2 wherein said heterocyclic ring comprises 9 to 15 ring atoms, at least 3 of said ring atoms being nitrogen.

7. The metal coordinating moiety of claim 6 wherein said heterocyclic ring comprises 3 to 5 ring nitrogen atoms.

8. The metal coordinating moiety of claim 6 wherein said heterocyclic ring is substituted at one or more ring carbon atoms, or at one more ring nitrogen atoms.

9. The metal coordinating moiety of claim 1 wherein the metal coordinating moiety comprises a substituted heterocyclic ring having the following structure (3), wherein: 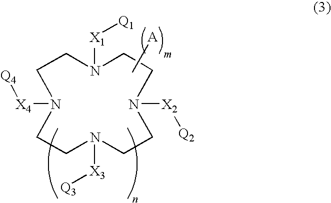
n is 0, 1 or 2;
m is 0-20, wherein when m is greater than 0, each A is independently C₁₋₂₀ alkyl or aryl optionally substituted by one or more aryl, C₁₋₂₀ alkyl, carbaldehyde, keto, carboxyl, cyano, halo, nitro, amido, polypeptides, sulfato, sulfito, phosphato, phosphito hydroxyl, oxy, ether, polyether, C₄₋₂₀ carbohydrate, mercapto or thio;
X₁, X₂, X₃ and X₄ are independently optionally substituted methylene where the substituents are selected from aryl, C₁₋₂₀ alkyl, carbaldehyde, keto, carboxyl, cyano, halo, nitro, amido, polypeptides, sulfate, sulfito, phosphato, phosphito, hydroxyl, oxy, ether, polyether, C₄₋₂₀ carbohydrate, mercapto or thio;
Q₁ is 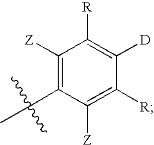
Q₂, Q₃ and Q₄ are independently selected from optionally substituted methylthio, carboxyl, phosphonate, sulfonate, and 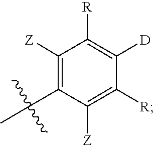
each Z is independently hydrogen, hydroxy, or thiol provided, however, that at least one Z substituent is other than hydrogen;
each R is independently bromo or iodo; and
D is selected from fluoro, chloro, bromo, iodo, carboxyl, cyano, nitro, amido, hydroxyl, amino, ammonium, sulfato, sulfito, phosphato, phosphito, ether, polyether, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito.

10. The metal coordinating moiety of claim 1 wherein the metal chelator comprises a substituted chain of carbon and nitrogen atoms.

11. The metal coordinating moiety of claim 10 wherein said substituted chain comprises 4 to 10 atoms, at least 2 of said atoms being nitrogen.

12. The metal coordinating moiety of claim 11 wherein said substituted chain comprises 2 to 4 nitrogen atoms.

13. The metal coordinating moiety of claim 10 wherein said substituted chain is substituted at one or more carbon atoms, or at one or more nitrogen atoms.

14. The metal coordinating moiety of claim 1 wherein the metal coordinating moiety comprises a substituted chain of carbon and nitrogen atoms having the following structure (4), wherein: 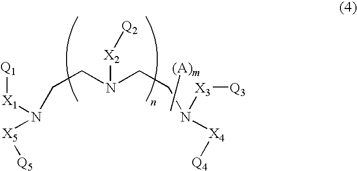
n is 0, 1 or 2;
m is 0-12, wherein when m is greater than 0, each A is independently C₁₋₂₀ alkyl or aryl optionally substituted by one or more aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto or thio,
X₁, X₂, X₃ and X₄ and X₅ are independently optionally substituted methylene where the substituents are selected from the group consisting of aryl, C₁₋₂₀ alkyl, carbaldehyde, keto, carboxyl, cyano, halo, nitro, amido, polypeptides, sulfato, sulfito, phosphato, phosphito, hydroxyl, oxy, ether, polyether, C₄₋₂₀ carbohydrate, mercapto and thio;
Q₁ is 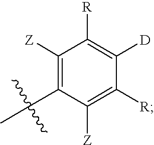
Q2, Q3, Q4 and Q5 are independently selected from optionally substituted methylthio, carboxyl, phosphonate, sulfonate, or 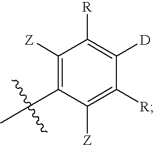
each Z is independently hydrogen, hydroxy, or thiol provided, however, that at least one Z substituent is other than hydrogen;
each R is independently bromo or iodo; and
D is selected from fluoro, chloro, bromo, iodo, carboxyl, cyano, nitro, amido, hydroxyl, amino, ammonium, sulfato, sulfito, phosphato, phosphito, ether, polyether, aryl, or C₁₋₂₀ alkyl optionally substituted with one or more of C₁₋₂₀ alkyl, carboxyl, cyano, nitro, amido, hydroxyl, amino, sulfato, sulfito, phosphato, and phosphito.

15. The metal coordinating moiety of claim 1 wherein the metal coordinating moiety comprises a halogen-substituted resorcinol derivative.

16. A pharmaceutical composition comprising the metal coordinating moiety of claim 4 and a pharmaceutically acceptable carrier.

17. A method of medical imaging comprising administering to a patient an effective amount of the metal coordinating moiety of claim 16.
